(19) United States
(12) Patent Application Publication
(10) Pub. No.: US 2007/0167430 A1
(43) Pub. Date: Jul. 19, 2007

(54) Compounds useful in therapy

(76) Inventor: Thomas Ryckmans, Kent (GB)

Correspondence Address: PFIZER INC, 150 EAST 42ND STREET, 5TH FLOOR - STOP 49, NEW YORK, NY, 10017-5612, US

(21) Appl. No.: 10/588,878

(22) PCT Filed: Jan. 5, 2005
(86) PCT No.: PCT/IB05/00263

§ 371 Date: Mar. 22, 2007

Related U.S. Application Data
(60) Provisional application No. 60/544,866, filed on Feb. 13, 2004.

(30) Foreign Application Priority Data
Jan. 13, 2004 (GB) 0400700.1

(51) Int. Cl. A61K 31/553; A61K 31/551; A61K 31/4162; C07D 491/04

(52) U.S. Cl. 514/211.12; 514/220; 514/406; 540/547; 540/557; 548/359.5

(57) ABSTRACT

Compounds of formula (I), or a pharmaceutically acceptable derivative thereof, wherein: X represents NR or O; R represents hydrogen, C₁₋₈ alkyl or SO₂└C,₁₋₈ alkyl┘; W represents N or CH; Y and Y′ independently represent hydrogen, halogen, OH, CF₃, OCF₃, CN, NH₂ C₁₋₈ alkyl, C₁₋₈ alkyloxy or C₃₋₈cycloalkyl; Ring A represents a heterocyclic ring containing at least one nitrogen atom; Z represents a direct link, C₁₋₈ alkyl or C₃₋₈ cycloalkyl; R¹ represents R², OR², OR³—R⁴, N(R²)[C₁₋₈ alkylene]ₐR⁴; NCOR², or SR⁴; R² and R⁴ independently represent hydrogen, C₃₋₈ cycloalkyl, CF₃, Ar or Het; R³ represents a direct link or C₁₋₈ alkyl; is 0 or 1; Ar represents an aromatic ring, optionally fused to a heterocyclic ring, and/or optionally substituted with one or more groups as described below; Het represents a heterocyclic ring optionally substituted with one or more groups as described below, and/or optionally fused to an aromatic ring which is optionally substituted with one or more groups as described below; at each occurrence C₁₋₈alkyl, C₁₋₈alkylene and C₃₋₈cycloalkyl may be independently optionally substituted with one or more groups as described below; substituent groups for Ar, Het, C₁₋₈alkyl, C₁₋₈alkylene and C₃₋₈cycloalkyl referred to the above are independently selected from hydrogen, halogen, C₁₋₈alkyl, C₁₋₈alkyloxy, S[C₁₋₈alkyl], CN, CF₃, NH₂ and OH; are useful for treating anxiety, cardiovascular disease (including angina, atherosclerosis, hypertension, heart failure, edema, hypernatremia), dysmenorrhoea (primary and secondary), endometriosis, emesis (including motion sickness), intrauterine growth retardation, inflammation (including rheumatoid arthritis), mittelschmerz, preclampsia, premature ejaculation, premature (preterm) labor and Raynaud's disease.

This invention relates to novel compounds useful in therapy. To processes for the preparation of such compounds and intermediates used in their preparation. It further relates to compositions containing such compounds and their use.

WO 01/87855 discloses triazole derivatives as inhibitors of glycine transporter activity. WO 01/58880 and JP2000-63363 disclose triazole derivatives useful as arginine Vasopressin V_{1A} receptor antagonists. Kakefuda et al., Bioorg. Med. Chem. 10 (2002) 1905-1912 and Kakefuda et al., J. Med. Chem., 2002, 45, 2589-2598 discuss the utility of 4,5-diphenyl-1,2,4-triazole derivatives as selective antagonists for the human V_{1A} receptor and comment that the 4,5-diphenyl-1,2,4-triazole structure plays an essential role in V_{1A} affinity.

The compounds of the present invention have been found to have useful pharmaceutical properties. They may be used to treat one or more diseases selected from aggression, Alzheimer's disease, anorexia nervosa, anxiety, anxiety disorder, asthma, atherosclerosis, autism, cardiovascular disease (including angina, atherosclerosis, hypertension, heart failure, edema, hypernatremia), cataract, central nervous system disease, cerebrovascular ischemia, cirrhosis, cognitive disorder, Cushing's disease, depression, diabetes mellitus, dysmenorrhoea (primary and secondary), emesis (including motion sickness), endometriosis, gastrointestinal disease, glaucoma, gynaecological disease, heart disease, intrauterine growth retardation, inflammation (including rheumatoid arthritis), ischemia, ischemic heart disease, lung tumor, micturition disorder, mittelschmerz, neoplasm, nephrotoxicity, non-insulin dependent diabetes, obesity, obsessive/compulsive disorder, ocular hypertension, preclampsia, premature ejaculation, premature (preterm) labor, pulmonary disease, Raynaud's disease, renal disease, renal failure, male or female sexual dysfunction, septic shock, sleep disorder, spinal cord injury, thrombosis, urogenital tract infection and urolithiasis.

Particularly of interest are the following diseases or disorders:
anxiety, cardiovascular disease (including angina, atherosclerosis, hypertension, heart failure, edema, hypernatremia), dysmenorrhoea (primary and secondary), endometriosis, emesis (including motion sickness), intrauterine growth retardation, inflammation (including rheumatoid arthritis), mittelschmerz, preclampsia, premature ejaculation, premature (preterm) labor and Raynaud's disease.

In particular, the compounds of the present invention exhibit vasopressin antagonistic activity and can be used in the treatment of dysmenorrhoea (primary and secondary).

There is a high unmet need in the area of menstrual disorders and it is estimated that up to 90% of all menstruating women are affected to some degree. Up to 42% of women miss work or other activities due to menstrual pain and it has been estimated that around 600 million work hours a year are lost in the US as a result {Coco, A. S. (1999). Primary dysmenorrhoea. [Review] [30 refs]. American Family Physician, 60, 489-96.}.

Menstrual pain in the lower abdomen is caused by myometrial hyperactivity and reduced uterine blood flow. These pathophysiological changes result in abdominal pain that radiates out to the back and legs. This may result in women feeling nauseous, having headaches and suffering from insomnia. This condition is called dysmenorrhoea and can be classified as either primary or secondary dysmenorrhoea.

Primary dysmenorrhoea is diagnosed when no abnormality causing the condition is identified. This affects up to 50% of the female population {Coco, A. S. (1999). Primary dysmenorrhoea. [Review] [30 refs]. American Family Physician, 60, 489-96.; Schroeder, B. & Sanfilippo, J. S. (1999). Dysmenorrhoea and pelvic pain in adolescents. [Review] [78 refs]. Pediatric Clinics of North America, 46, 555-71}. Where an underlying gynaecological disorder is present, such as endometriosis, pelvic inflammatory disease (PID), fibroids or cancers, secondary dysmenorrhoea will be diagnosed. Secondary dysmenorrhoea is diagnosed in only approximately 25% of women suffering from dysmenorrhoea. Dysmenorrhoea can occur in conjunction with menorrhagia, which accounts for around 12% of referrals to gynaecology outpatients departments.

Currently, women suffering from primary dysmenorrhoea are treated with non-steroidal anti-inflammatory drugs (NSAID's) or the oral contraceptive pill. In cases of secondary dysmenorrhoea surgery may be undertaken to correct the underlying gynaecological disorder.

Women suffering from dysmenorrhoea have circulating vasopressin levels which are greater than those observed in healthy women at the same time of the menstrual cycle. Inhibition of the pharmacological actions of vasopressin, at the uterine vasopressin receptor, may prevent dysmenorrhoea.

According to the present invention there is provided a compound of formula (I), 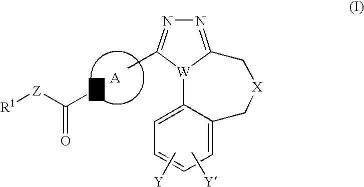
or a pharmaceutically acceptable derivative thereof, wherein:
X represents NR or O;
   R represents hydrogen, C₁₋₈ alkyl or SO₂[C₁₋₈ alkyl];
W represents N or CH;
Y and Y′ independently represent hydrogen, halogen, OH, CF₃, OCF₃, CN, NH₂, C₁₋₈ alkyl, C₁₋₈ alkyloxy or C₃₋₈ cycloalkyl;
Ring A represents a heterocyclic ring containing at least one nitrogen atom;
Z represents a direct link, C₁₋₈ alkyl or C₃₋₈ cycloalkyl;
R¹ represents R², OR², OR³—R⁴, N(R²)[C₁₋₈ alkylene]ₐR⁴; NCOR², or SR⁴;
   R² and R⁴ independently represent hydrogen, C₃₋₈ cycloalkyl, CF₃, Ar or Het;
   R³ represents a direct link or C₁₋₈ alkyl;
   a is 0 or 1;
      Ar represents an aromatic ring, optionally fused to a heterocyclic ring, and/or optionally substituted with one or more groups as described below;
      Het represents a heterocyclic ring optionally substituted with one or more groups as described below, and/or optionally fused to an aromatic ring which is optionally substituted with one or more groups as described below;
         at each occurrence C₁₋₈alkyl, C₁₋₈alkylene and C₃₋₈cycloalkyl may be independently optionally substituted with one or more groups as described below;
   substituent groups for Ar, Het, C₁₋₈alkyl, C₁₋₈alkylene and C₃₋₈cycloalkyl referred to above are independently selected from hydrogen, halogen, C₁₋₈alkyl, C₁₋₈alkyloxy, S[C₁₋₈alkyl], CN, CF₃, NH₂ and OH.

In the above definitions, halogen means fluoro, chloro, bromo or iodo. Alkyl and alkyloxy, groups containing the requisite number of carbon atoms, except where indicated, can be unbranched or branched chain. Examples of alkyl include methyl, ethyl, n-propyl, i-propyl, n-butyl, i-butyl, sec-butyl and t-butyl. Examples of alkoxy include methoxy, ethoxy, n-propoxy, i-propoxy, n-butoxy, i-butoxy, sec-butoxy and t-butoxy. Examples of cycloalkyl include cyclopropyl, cyclobutyl, cyclopentyl, cyclohexyl and cycloheptyl.

Unless otherwise stated, the term heterocyclic ring, or heterocyclic, means a five- or six-membered saturated, unsaturated or aromatic ring containing one or more heteroatoms selected from N, S and O. Preferred heterocycles included within the definition of “heterocycle” are pyrrolyl, imidazolyl, triazolyl, thienyl, furyl, thiazolyl, oxazolyl, thiadiazolyl, oxadiazolyl, pyridinyl, pyrimidinyl, pyridazinyl, pyrazinyl, indolyl, isoindolyl, quinolinyl, isoquinolinyl, benzimidazolyl, quinazolinyl, phthalazinyl, benzoxazolyl and quinoxalinyl, together with partially or fully saturated versions thereof as well as azetidinyl, pyrrolidinyl, piperidinyl, piperazinyl, homopiperazinyl and morpholinyl.

The term aryl ring means a five- or six-membered aromatic ring.

Preferred groups of compounds are those in which one or more of the following apply:
(i) X represents NR;
(ii) R represents Me;
(iii) W represents N;
(iv) Ring A represents piperidinyl;
(v) Z represents a direct link;
(vi) R¹ represents a phenyl ring substituted with halogen and/or alkyl;
(vii) R¹ represents a phenyl ring fused to a five-membered, nitrogen containing heterocycle.

Preferred compounds according to the present invention are:
[4-(8-Chloro-5-methyl-5,6-dihydro-4H-2,3,5,10b-tetraaza-benzo[e]azulen-1-yl)-piperidin-1-yl]-(1H-indol-3-yl)-methanone;
1-[4-(8-Chloro-5-methyl-5,6-dihydro-4H-2,3,5,10b-tetraaza-benzo[e]azulen-1-yl)-piperidin-1-yl]-2-o-tolyl-ethanone;
[4-(8-Chloro-5-methyl-5,6-dihydro-4H-2,3,5,10b-tetraaza-benzo[e]azulen-1-yl)-piperidin-1-yl]-(1-methyl-cyclohexyl)-methanone;
1-[4-(8-Chloro-5-methyl-5,6-dihydro-4H-2,3,5,10b-tetraaza-benzo[e]azulen-1-yl)-piperidin-1-yl]-2-cyclopropyl-ethanone;
[4-(8-Chloro-5-methyl-5,6-dihydro-4H-2,3,5,10b-tetraaza-benzo[e]azulen-1-yl)-piperidin-1-yl]-(1H-indol-2-yl)-methanone;
[4-(8-Chloro-5-methyl-5,6-dihydro-4H-2,3,5,10b-tetraaza-benzo[e]azulen-1-yl)-piperidin-1-yl]-(2-hydroxy-5-methyl-phenyl)-methanone;
[4-(8-Chloro-5-methyl-5,6-dihydro-4H-2,3,5,10b-tetraaza-benzo[e]azulen-1-yl)-piperidin-1-yl]-(1H-indol-6-yl)-methanone;
[4-(8-Chloro-5-methyl-5,6-dihydro-4H-2,3,5,10b-tetraaza-benzo[e]azulen-1-yl)-piperidin-1-yl]-(3-methoxy-phenyl)-methanone;
[4-(8-Chloro-5-methyl-5,6-dihydro-4H-2,3,5,10b-tetraaza-benzo[e]azulen-1-yl)-piperidin-1-yl]-(3-fluoro-phenyl)-methanone;
[4-(8-Chloro-5-methyl-5,6-dihydro-4H-2,3,5,10b-tetraaza-benzo[e]azulen-1-yl)-piperidin-1-yl]-(4-fluoro-phenyl)-methanone;
1-[4-(8-Chloro-5-methyl-5,6-dihydro-4H-2,3,5,10b-tetraaza-benzo[e]azulen-1-yl)-piperidin-1-yl]-butan-1-one;
[4-(8-Chloro-5-methyl-5,6-dihydro-4H-2,3,5,10b-tetraaza-benzo[e]azulen-1-yl)-piperidin-1-yl]-cyclopropyl-methanone; and
pharmaceutically acceptable derivatives thereof.

Pharmaceutically acceptable derivatives of the compounds of formula (I) according to the invention include salts, solvates, complexes, polymorphs, prodrugs, stereoisomers, geometric isomers, tautomeric forms, and isotopic variations of compounds of formula (I). Preferably, pharmaceutically acceptable derivatives of compounds of formula (I) comprise salts, solvates, esters and amides of the compounds of formula (I). More preferably, pharmaceutically acceptable derivatives of compounds of formula (I) are salts and solvates.

The pharmaceutically acceptable salts of the compounds of formula (I) include the acid addition and base salts thereof.

Suitable acid addition salts are formed from acids which form non-toxic salts. Examples include the acetate, aspartate, benzoate, besylate, bicarbonate/carbonate, bisulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, D- and L-lactate, malate, maleate, malonate, mesylate, methylsulphate, naphthylate, 2-napsylate, nicotinate, nitrate, orotate, oxalate, palmitate, palmoate, phosphate, hydrogen phosphate, dihydrogen phosphate, saccharate, stearate, succinate, sulphate, D- and L-tartrate, tosylate and trifluoroacetate salts. A particularly suitable salt is the besylate derivative of the compounds of the present invention.

Suitable base salts are formed from bases, which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

For a review on suitable salts see Stahl and Wermuth, Handbook of Pharmaceutical Salts: Properties, Selection and Use, Wiley-VCH, Weinheim, Germany (2002).

A pharmaceutically acceptable salt of a compound of formula (I) may be readily prepared by mixing together solutions of the compound of formula (I) and the desired acid or base, as appropriate. The salt may precipitate from solution and be collected by filtration or may be recovered by evaporation of the solvent. The degree of ionisation in the salt may vary from completely ionised to almost non-ionised.

The compounds of the invention may exist in both unsolvated and solvated forms. The term “solvate” is used herein to describe a molecular complex comprising the compound of the invention and one or more pharmaceutically acceptable solvent molecules, for example, ethanol. The term “hydrate” is employed when said solvent is water. Included within the scope of the invention are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components what may be in stoichiometric or non-stoichiometric amounts. The resulting complexes may be ionised, partially ionised, or non-ionised. For a review of such complexes, see J Pharm Sci, 64 (8), 1269-1288 by Haleblian (August 1975).

Hereinafter all references to compounds of formula (I) and pharmaceutically acceptable derivatives include references to salts, solvates and complexes thereof and to solvates and complexes of salts thereof.

The compounds of the invention include compounds of formula (I) as hereinbefore defined, polymorphs, prodrugs, and isomers thereof (including optical, geometric and tautomeric isomers) as hereinafter defined and isotopically-labelled compounds of formula (I).

As stated, the invention includes all polymorphs of the compounds of formula (I) as hereinbefore defined.

Also within the scope of the invention are so-called “prodrugs” of the compounds of formula (I). Thus certain derivatives of compounds of formula (I) which may have little or no pharmacological activity themselves can, when administered into or onto the body, be converted into compounds of formula (I) having the desired activity, for example, hydrolytic cleavage. Such derivatives are referred to as “prodrugs”. Further information on the use of prodrugs may be found in “Pro-drugs as Novel Delivery Systems, Vol. 14, ACS Symposium Series (T Higuchi and W Stella) and “Bioreversible Carriers in Drug Design”, Pergamon Press, 1987 (ed. E B Roche, American Pharmaceutical Association).

Prodrugs in accordance with the invention can, for example, be produced by replacing appropriate functionalities present in the compounds of formula (I) with certain moieties known to those skilled in the art as “pro-moieties” as described, for example, in “Design of Prodrugs” by H Bundgaard (Elsevier, 1985).

Some examples of prodrugs in accordance with the invention include:
(i) where the compound of formula (I) contains a carboxylic acid functionality (—COOH), an ester thereof, for example, replacement of the hydrogen with (C₁-C₈)alkyl;
(ii) where the compound of formula (I) contains an alcohol functionality (—OH), an ether thereof, for example, replacement of the hydrogen with (C₁-C₆)alkanoyloxymethyl; and
(iii) where the compound of formula (I) contains a primary or secondary amino functionality (—NH₂ or —NHR where R≠H), an amide thereof, for example, replacement of one or both hydrogens with (C₁-C₁₀)alkanoyl.

Further examples of replacement groups in accordance with the foregoing examples and examples of other prodrug types may be found in the aforementioned references.

Finally, certain compounds of formula (I) may themselves act as prodrugs of other compounds of formula (I).

Also within the scope of the invention are the metabolites of the compounds of formula (I) when formed in vivo.

Compounds of formula (I) containing one or more asymmetric carbon atoms can exist as two or more stereoisomers. Where a compound of formula (I) contains an alkenyl or alkenylene group, geometric cis/trans (or Z/E) isomers are possible, and where the compound contains, for example, a keto or oxime group or an aromatic moiety, tautomeric isomerism (‘tautomerism’) may occur. It follows that a single compound may exhibit more than one type of isomerism.

Included within the scope of the present invention are all stereoisomers, geometric isomers and tautomeric forms of the compounds of formula (I), including compounds exhibiting more than one type of isomerism, and mixtures of one or more thereof. Also included are acid addition or base salts wherein the counter ion is optically active, for example, D-lactate or L-lysine, or racemic, for example, DL-tartrate or DL-arginine.

Cis/trans isomers may be separated by conventional techniques well known to those skilled in the art, for example, fractional crystallisation and chromatography. Conventional techniques for the preparation/isolation of individual enantiomers include chiral synthesis from a suitable optically pure precursor or resolution of the racemate (or the racemate of a salt or derivative) using, for example, chiral HPLC.

Alternatively, the racemate (or racemic precursor) may be reacted with a suitable optically active compound, for example, an alcohol, or, in the case where the compounds of formula (I) contains an acidic or basic moiety, an acid or base such as tartaric acid or 1-phenylethylamine. The resulting diastereomeric mixture may be separated by chromatography and/or fractional crystallisation and one or both of the diastereomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Stereoisomeric conglomerates may be separated by conventional techniques known to those skilled in the art—see, for example, “Stereochemistry of Organic Compounds” by E L Eliel (Wiley, New York, 1994).

The present invention also includes all pharmaceutically acceptable isotopic variations of a compound of the formula (I) one or more atoms is replaced by atoms having the same atomic number, but an atomic mass or mass number different from the atomic mass or mass number usually found in nature.

Examples of isotopes suitable for inclusion in the compounds of the invention include isotopes of hydrogen such as ²H and ³H, carbon such as ¹¹C, ¹³C and ¹⁴C, nitrogen such as ¹³N and ¹⁵N, oxygen such as ¹⁵O, ¹⁷O and ¹⁸O, phosphorus such as ³²P, sulphur such as ³⁵S, fluorine such as ¹⁸F, iodine such as ¹²³I and ¹²⁵I, and chlorine such as ³⁶Cl.

Certain isotopically-labelled compounds of formula (I), for example those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labelled compounds of formula (I) can generally be prepared by conventional techniques known to those skilled in the art or by processes analogous to those described in the accompanying Examples and Preparations using appropriate isotopically-labelled reagents in place of the non-labelled reagent previously employed.

Pharmaceutically acceptable solvates in accordance with the invention include those wherein the solvent of crystallisation may be isotopically substituted, e.g. D₂O, d₆-acetone and d₆-DMSO.

Unless otherwise provided herein:
HBTU means O-benzotriazol-1-yl-N,N,N′,N′-tetramethyluronium hexafluoro phosphate;
Et₃N means triethylamine;
AcOH means acetic acid, TFA means trifluoroacetic acid;
MeOH means methanol, EtOH means ethanol, and EtOAc means ethyl acetate;
THF means tetrahydrofuran, DMSO means dimethyl sulphoxide, and DCM means dichloromethane, DMF means N,N-dimethylformamide, NMP means N-methyl-2-pyrrolidinone, DMA means dimethylacetamide;
Boc means tert-butoxy carbonyl, CBz means benzyloxy carbonyl;
Triflic anhydride means trifluoromethanesulphonic anhydride;
p-TSA means p-toluenesulphonic acid;
Dba means dibenzylideneacetone;
Me means methyl, Et means ethyl; Cl means chloro; OH means hydroxy;
LG means a suitable leaving group; and
Prot means a suitable protecting group.

The following schemes illustrate the preparation of compounds of the formula (I), throughout which Ring A etc. are as hereinbefore defined:

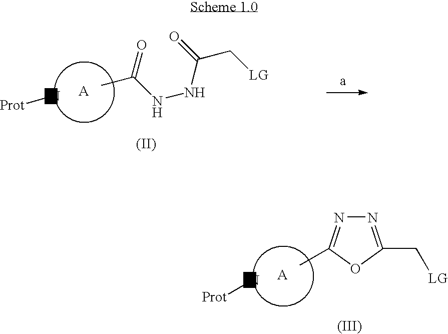

Compound (II) is described in International Patent Publication No. WO 97/03986.
Step (a): Cyclisation of compound (II) is carried out under suitable dehydrating conditions, at elevated temperatures for up to 18 hours.

Typically, dehydrating agents such as polyphosphoric acid, phosphorous oxychloride, triflic anhydride are used at temperatures from 20 to 120° C. for 5 minutes to 12 hours. Optionally, the reaction can be carried out in the presence of a base such as pyridine and suitable solvents such as dichloromethane and acetonitrile. Alternatively, the oxadiazole (III) may be prepared according to the method of Rigo et. al. Synth. Commun. 16(13), 1665, 1986.

Preferred conditions are:

Phosphorous oxychloride at 100° C. for 8 hours, or 2.5 eq. triflic anhydride, 5 eq. pyridine in dichloromethane at 20° C. for 3 hours.

When X represents O, then

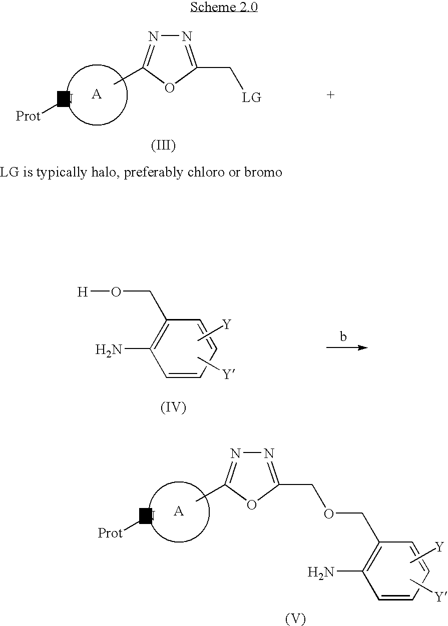

Prot represents a suitable protecting group for nitrogen, for example Boc, CBz or Allyl carbamate. Standard methodology for nitrogen protecting groups is used, such as that found in textbooks (e.g. “Protecting Groups in Organic Synthesis” by T. W. Greene and P. Wutz). Compounds suitable for use as compounds (IV) are known in the literature or can be prepared using standard methodology: for example, reduction of benzoic acids (see preparation 3 below).
Step (b): Compound (III) is reacted with an excess of compound (IV) in the presence of a base such as sodium hydride, potassium hexamethyldisilazide, ⁿbutyl lithium or isopropyl magnesium chloride, in a suitable solvent such as THF, Toluene or NMP at temperatures from 0° C. to 50° C. for 1 to 24 hours, to give compound (V) respectively.

Preferred conditions are:

3 eq. of compound (IV) and 2.5 eq. of NaH in THF at 20° C. for 2 hours.

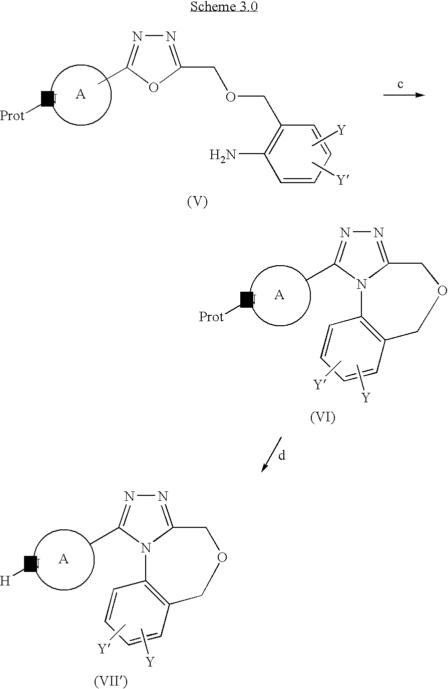

Prot represents a suitable protecting group for nitrogen, for example Boc, CBz or Allyl carbamate. Standard methodology for nitrogen protecting groups is used, such as that found in textbooks (e.g. “Protecting Groups in Organic Synthesis” by T. W. Greene and P. Wutz).
Step (c): Oxadiazole (V) is reacted with an acid catalyst to give the compound of formula (VI). Typically the reaction is carried out by heating the starting materials with a suitable acidic catalyst (such as p-TSA), or Lewis acid catalyst (such as magnesium chloride), optionally using a high boiling solvent such as xylene, at an elevated temperature, such as 100-150° C., for 1 to 48 hours. Alternatively, the reaction may be subjected to microwave irradiation and heated to 150-200° C. for 10-30 minutes in a high boiling point solvent such as toluene or xylene.

Preferred conditions are:
Oxadiazole (V) and cat. P-TSA, in xylene at 140° C. for 48 hrs.
Step (d): Deprotection of compound (VI) is undertaken using standard methodology, as described in “Protecting Groups in Organic Synthesis” by T. W. Greene and P. Wutz”.

When Prot is Boc, the preferred methods are:
hydrogen chloride in a suitable solvent such as 1,4-dioxane at room temperature for 1-16 hours; or
a solution of trifluoroacetic acid in dichloromethane for 1-2 hours.

When Prot is CBz, the preferred method is hydrogenolysis using a suitable palladium catalyst in a solvent such as ethanol.

When Prot is an allyl carbamate, preferred conditions are thiobenzoic acid and a suitable palladium catalyst such as Pd₂(Dba)₃ with a suitable phosphine additive such as 1,4-bis(diphenylphosphino)butane in tetrahydrofuran for 20 minutes.

When X represents N-alkyl, then 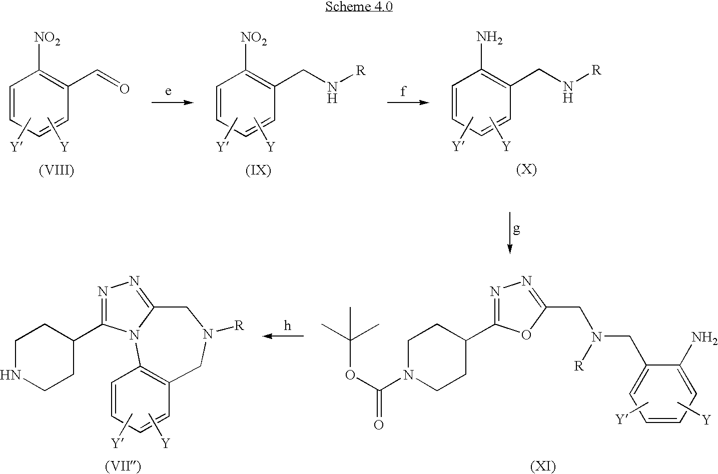
Step (e): Commercially available 2-nitrobenzaldehyde (VIII) is reacted with methylamine hydrochloride to give the compound of formula (IX). Typically the reaction is carried out in methylene chloride in the presence of triethylamine for 16 hours.
Step (f): Amine (IX) in ethanol is reacted with hydrogen in the presence of PtO₂ under 40 PSI for 2 hours to give compound (X).
Step (g): Amine (X) is reacted with oxadiazole (III) to give a compound of formula (XI). Typically the reaction is carried out by refluxing the starting materials in THF, in the presence of triethylamine, for 24 hours.
Step (h): Oxadiazole (XI) in toluene is reacted with TFA at 50° C. for 1 hour to give a compound of formula (VII″).

Compounds (VII′) and (VII″) correspond to compounds of formula (VII) when X represents O and NR respectively.

Compounds of formula (VII) are used in a chemical library synthesis to provide compounds of formula (I):

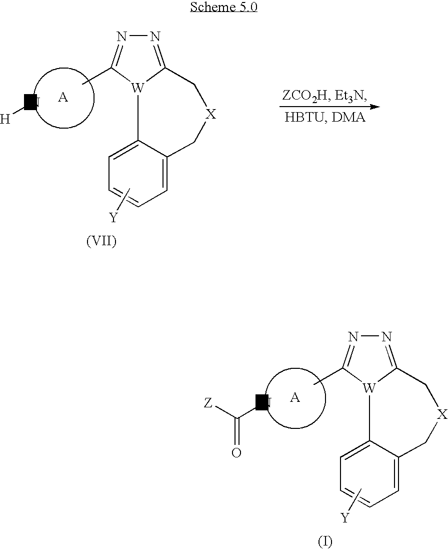

Reactions are run in DMA, and a standard peptide coupling reagent (HBTU) is used to bring about the amide bond formation.

The compounds of the present invention are useful because they possess pharmacological activity in animals. In particular they are useful in the treatment of a number of conditions including aggression, Alzheimer's disease, anorexia nervosa, anxiety, anxiety disorder, asthma, atherosclerosis, autism, cardiovascular disease (including angina, atherosclerosis, hypertension, heart failure, edema, hypernatremia), cataract, central nervous system disease, cerebrovascular ischemia, cirrhosis, cognitive disorder, Cushing's disease, depression, diabetes mellitus, dysmenorrhoea (primary and secondary), emesis (including motion sickness), endometriosis, gastrointestinal disease, glaucoma, gynaecological disease, heart disease, intrauterine growth retardation, inflammation (including rheumatoid arthritis), ischemia, ischemic heart disease, lung tumor, micturition disorder, mittelschmerz, neoplasm, nephrotoxicity, non-insulin dependent diabetes, obesity, obsessive/compulsive disorder, ocular hypertension, preclampsia, premature ejaculation, premature (preterm) labor, pulmonary disease, Raynaud's disease, renal disease, renal failure, male or female sexual dysfunction, septic shock, sleep disorder, spinal cord injury, thrombosis, urogenital tract infection or urolithiasis.sleep disorder, spinal cord injury, thrombosis, urogenital tract infection, urolithiasis. Particularly of interest is dysmenorrhoea (primary or secondary), more particularly, primary dysmenorrhoea.

Thus, according to another aspect of the invention, there is provided a method of treatment of dysmenorrhoea which comprises administering a therapeutically effective amount of a compound of the invention to a patient suffering from anxiety, cardiovascular disease (including angina, atherosclerosis, hypertension, heart failure, edema, hypernatremia), dysmenorrhoea (primary and secondary), endometriosis, emesis (including motion sickness), intrauterine growth retardation, inflammation (including rheumatoid arthritis), mittelschmerz, preclampsia, premature ejaculation, premature (preterm) labor or Raynaud's disease. The use of the compounds as a medicament and the use of the compounds of the present invention in the manufacture of a medicament for the treatment of anxiety, cardiovascular disease (including angina, atherosclerosis, hypertension, heart failure, edema, hypernatremia), dysmenorrhoea (primary and secondary), endometriosis, emesis (including motion sickness), intrauterine growth retardation, inflammation (including rheumatoid arthritis), mittelschmerz, preclampsia, premature ejaculation, premature (preterm) labor or Raynaud's disease, particularly dysmenorrhoea, are also provided.

Compounds of the invention intended for pharmaceutical use may be administered as crystalline or amorphous products. They may be obtained, for example, as solid plugs, powders, or films by methods such as precipitation, crystallisation, freeze drying, spray drying, or evaporative drying. Microwave or radio frequency drying may be used for this purpose.

They may be administered alone or in combination with one or more other compounds of the invention or in combination with one or more other drugs (or as any combination thereof). The compounds of the present invention may be administered in combination with an oral contraceptive. Thus in a further aspect of the invention, there is provided a pharmaceutical product containing an V1a antagonist and an oral contraceptive as a combined preparation for simultaneous, separate or sequential use in the treatment of dysmenorrhoea.

The compounds of the present invention may be administered in combination with a PDE5 inhibitor. Thus in a further aspect of the invention, there is provided a pharmaceutical product containing a V1a antagonist and a PDEV inhibitor as a combined preparation for simultaneous, separate or sequential use in the treatment of dysmenorrhoea.

PDEV inhibitors useful for combining with V1a antagonists include, but are not limited to:
(i) The PDE5 inhibitors mentioned in International Patent Application publication nos. WO03/000691; WO02/64590; WO02/28865; WO02/28859; WO02/38563; WO02/36593; WO02/28858; WO02/00657; WO02/00656; WO02/10166; WO02/00658; WO01/94347; WO01/94345; WO00/15639 and WO00/15228;
(ii) The PDE5 inhibitors mentioned in U.S. Pat. Nos. 6,143,746; 6,143,747 and 6,043,252;
(iii) the pyrazolo [4,3-d]pyrimidin-7-ones disclosed in EP-A-0463756; the pyrazolo [4,3-d]pyrimidin-7-ones disclosed in EP-A-0526004; the pyrazolo [4,3-d]pyrimidin-7-ones disclosed in published international patent application WO 93/06104; the isomeric pyrazolo [3,4-d]pyrimidin-4-ones disclosed in published international patent application WO 93/07149; the quinazolin-4-ones disclosed in published international patent application WO 93/12095; the pyrido [3,2-d]pyrimidin-4-ones disclosed in published international patent application WO 94/05661; the purin-6-ones disclosed in published international patent application WO 94/00453; the pyrazolo [4,3-d]pyrimidin-7-ones disclosed in published international patent application WO 98/49166; the pyrazolo [4,3-d]pyrimidin-7-ones disclosed in published international patent application WO 99/54333; the pyrazolo [4,3-d]pyrimidin-4-ones disclosed in EP-A-0995751; the pyrazolo [4,3-d]pyrimidin-7-ones disclosed in published international patent application WO 00/24745; the pyrazolo [4,3-d]pyrimidin-4-ones disclosed in EP-A-0995750; the hexahydropyrazino [2′,1′:6,1]pyrido [3,4-b]indole-1,4-diones disclosed in published international application WO95/19978; the pyrazolo [4,3-d]pyrimidin-4-ones disclosed in WO00/27848; the imidazo[5,1-f][1,2,4]triazin-ones disclosed in EP-A-1092719 and in published international application WO 99/24433 and the bicyclic compounds disclosed in published international application WO 93/07124; the pyrazolo [4,3-d]pyrimidin-7-ones disclosed in published international application WO 01/27112; the pyrazolo [4,3-d]pyrimidin-7-ones disclosed in published international application WO 01/27113; the compounds disclosed in EP-A-1092718 and the compounds disclosed in EP-A-1092719; the tricyclic compounds disclosed in EP-A-1241170; the alkyl sulphone compounds disclosed in published international application WO 02/074774; the compounds disclosed in published international application WO 02/072586; the compounds disclosed in published international application WO 02/079203 and the compounds disclosed in WO 02/074312.
(iv) Preferably 5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil, e.g. as sold as Viagra®) also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine (see EP-A-0463756); 5-(2-ethoxy-5-morpholinoacetylphenyl)-1-methyl-3-n-propyl-1,6-dihydro-7H-pyrazolo[4,3-d]pyrimidin-7-one (see EP-A-0526004); 3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one (see WO98/49166); 3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one (see WO99/54333); (+)-3-ethyl-5-[5-(4-ethylpiperazin-1-ylsulphonyl)-2-(2-methoxy-1(R)-methylethoxy)pyridin-3-yl]-2-methyl-2,6-dihydro-7H-pyrazolo[4,3-d]pyrimidin-7-one, also known as 3-ethyl-5-{5-[4-ethylpiperazin-1-ylsulphonyl]-2-([(1R)-2-methoxy-1-methylethyl]oxy)pyridin-3-yl}-2-methyl-2,6-dihydro-7H-pyrazolo[4,3-d]pyrimidin-7-one (see WO99/54333); 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, also known as 1-{6-ethoxy-5-[3-ethyl-6,7-dihydro-2-(2-methoxyethyl)-7-oxo-2H-pyrazolo[4,3-d]pyrimidin-5-yl]-3-pyridylsulphonyl}-4-ethylpiperazine (see WO 01/27113, Example 8); 5-[2-iso-Butoxy-5-(4-ethylpiperazin-1-ylsulphonyl)pyridin-3-yl]-3-ethyl-2-(1-methylpiperidin-4-yl)-2,6-dihydro-7H-pyrazolo[4,3-d]pyrimidin-7-one (see WO 01/27113, Example 15); 5-[2-Ethoxy-5-(4-ethylpiperazin-1-ylsulphonyl)pyridin-3-yl]-3-ethyl-2-phenyl-2,6-dihydro-7H-pyrazolo[4,3-d]pyrimidin-7-one (see WO 01/27113, Example 66); 5-(5-Acetyl-2-propoxy-3-pyridinyl)-3-ethyl-2-(1-isopropyl-3-azetidinyl)-2,6-dihydro-7H-pyrazolo[4,3d]-pyrimidin-7-one (see WO 01/27112, Example 124); 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one (see WO 01/27112, Example 132); (6R,12aR)-2,3,6,7,12,12a-hexahydro-2-methyl-6-(3,4-methylenedioxyphenyl)pyrazino[2′,1′:6,1]pyrido[3,4-b]indole-1,4-dione (tadalafil, IC-351, Cialis®), i.e. the compound of examples 78 and 95 of published international application WO95/19978, as well as the compound of examples 1, 3, 7 and 8; 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil, LEVITRA®) also known as 1-[[3-(3,4-dihydro-5-methyl-4-oxo-7-propylimidazo[5,1-f]-as-triazin-2-yl)-4-ethoxyphenyl]sulphonyl]-4-ethylpiperazine, i.e. the compound of examples 20, 19, 337 and 336 of published international application WO99/24433; the compound of example 11 of published international application WO93/07124 (EISAI); compounds 3 and 14 from Rotella D P, J. Med. Chem., 2000, 43, 1257; 4-(4-chlorobenzyl)amino-6,7,8-trimethoxyquinazoline; N-[[3-(4,7-dihydro-1-methyl-7-oxo-3-propyl-1H-pyrazolo[4,3-d]-pyrimidin-5-yl)-4-propxyphenyl]sulfonyl]-1-methyl2-pyrrolidinepropanamide [“DA-8159” (Example 68 of WO00/27848)]; and 7,8-dihydro-8-oxo-6-[2-propoxyphenyl]-1H-imidazo[4,5-g]quinazoline and 1-[3-[1-[(4-fluorophenyl)methyl]-7,8-dihydro-8-oxo-1H-imidazo[4,5-g]quinazolin-6-yl]-4-propoxyphenyl]carboxamide.
(v) 4-bromo-5-(pyridylmethylamino)-6-[3-(4-chlorophenyl)-propoxy]-3(2H)pyridazinone; 1-[4-[(1,3-benzodioxol-5-ylmethyl)amiono]-6-chloro-2-quinozolinyl]-4-piperidine-carboxylic acid, monosodium salt; (+)-cis-5,6a,7,9,9,9a-hexahydro-2-[4-(trifluoromethyl)-phenylmethyl-5-methyl-cyclopent-4,5]imidazo[2,1-b]purin-4(3H)one; furazlocillin; cis-2-hexyl-5-methyl-3,4,5,6a,7,8,9,9a-octahydrocyclopent[4,5]-imidazo[2,1-b]purin-4-one; 3-acetyl-1-(2-chlorobenzyl)-2-propylindole-6-carboxylate; 3-acetyl-1-(2-chlorobenzyl)-2-propylindole-6-carboxylate; 4-bromo-5-(3-pyridylmethylamino)-6-(3-(4-chlorophenyl)propoxy)-3-(2H)pyridazinone; I-methyl-5(5-morpholinoacetyl-2-n-propoxyphenyl)-3-n-propyl-1,6-dihydro-7H-pyrazolo(4,3-d)pyrimidin-7-one; 1-[4-[(1,3-benzodioxol-5-ylmethyl)arnino]-6-chloro-2-quinazolinyl]-4-piperidinecarboxylic acid, monosodium salt; Pharmaprojects No. 4516 (Glaxo Wellcome); Pharmaprojects No. 5051 (Bayer); Pharmaprojects No. 5064 (Kyowa Hakko; see WO 96/26940); Pharmaprojects No. 5069 (Schering Plough); GF-196960 (Glaxo Wellcome); E-8010 and E-4010 (Eisai); Bay-38-3045 & 38-9456 (Bayer); FR229934 and FR226807 (Fujisawa); and Sch-51866.

The contents of the published patent applications and journal articles and in particular the general formulae of the therapeutically active compounds of the claims and exemplified compounds therein are incorporated herein in their entirety by reference thereto.

Preferably the PDEV inhibitor is selected from sildenafil, tadalafil, vardenafil, DA-8159 and 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one.

Most preferably the PDE5 inhibitor is sildenafil and pharmaceutically acceptable salts thereof. Sildenafil citrate is a preferred salt.

The compounds of the present invention may be administered in combination with an NO donor. Thus in a further aspect of the invention, there is provided a pharmaceutical product containing a V1a antagonist and a NO donor as a combined preparation for simultaneous, separate or sequential use in the treatment of dysmenorrhoea.

The compounds of the present invention may be administered in combination with L-arginine, or as an arginate salt. Thus in a further aspect of the invention, there is provided a pharmaceutical product containing a V1a antagonist and L-arginine as a combined preparation for simultaneous, separate or sequential use in the treatment of dysmenorrhoea.

The compounds of the present invention may be administered in combination with a COX inhibitor. Thus in a further aspect of the invention, there is provided a pharmaceutical product containing a V1a antagonist and a COX inhibitor as a combined preparation for simultaneous, separate or sequential use in the treatment of dysmenorrhoea.

COX inhibitors useful for combining with the compounds of the present invention include, but are not limited to:
(i) ibuprofen, naproxen, benoxaprofen, flurbiprofen, fenoprofen, fenbufen, ketoprofen, indoprofen, pirprofen, carprofen, oxaprozin, prapoprofen, miroprofen, tioxaprofen, suprofen, alminoprofen, tiaprofenic acid, fluprofen, bucloxic acid, indomethacin, sulindac, tolmetin, zomepirac, diclofenac, fenclofenec, aiclofenac, ibufenac, isoxepac, furofenac, tiopinac, zidometacin, acetyl salicylic acid, indometacin, piroxicam, tenoxicam, nabumetone, ketorolac, azapropazone, mefenamic acid, tolfenamic acid, diflunisal, podophyllotoxin derivatives, acemetacin, droxicam, floctafenine, oxyphenbutazone, phenylbutazone, proglumetacin, acemetacin, fentiazac, clidanac, oxipinac, mefenamic acid, meclofenamic acid, flufenamic acid, niflumic acid, flufenisal, sudoxicam, etodolac, piprofen, salicylic acid, choline magnesium trisalicylate, salicylate, benorylate, fentiazac, clopinac, feprazone, isoxicam and 2-fluoro-a-methyl[1,1′-biphenyl]-4-acetic acid, 4-(nitrooxy)butyl ester (See Wenk, et al., Europ. J. Pharmacol. 453:319-324 (2002));
(ii) meloxicam, (CAS registry number 71125-38-7; described in U.S. Pat. No. 4,233,299), or a pharmaceutically acceptable salt or prodrug thereof;
(iii) Substituted benzopyran derivatives that are described in U.S. Pat. No. 6,271,253. Also benzopyran derivatives described in U.S. Pat. Nos. 6,034,256 and 6,077,850 along with International Publication No's WO 98/47890 and WO 00/23433;
(iv) Chromene COX2 selective inhibitors described in U.S. Pat. Nos. 6,077,850 and 6,034,256;
(v) The compounds described in International Patent Application Publication No's WO 95/30656, WO 95/30652, WO 96/38418 and WO 96/38442, and the compounds described in European Patent Application Publication No. 799823, along with the pharmaceutically acceptable derivatives thereof;
(vi) celecoxib (U.S. Pat. No. 5,466,823), valdecoxib (U.S. Pat. No. 5,633,272), deracoxib (U.S. Pat. No. 5,521,207), rofecoxib (U.S. Pat. No. 5,474,995), etoricoxib (International Patent Application Publication No. WO 98/03484), JTE-522 (Japanese Patent Application Publication No. 9052882), or a pharmaceutically acceptable salt or prodrug thereof;
(vii) Parecoxib (described in U.S. Pat. No. 5,932,598), which is a therapeutically effective prodrug of the tricyclic Cox-2 selective inhibitor valdecoxib (described in U.S. Pat. No. 5,633,272), in particular sodium parecoxib;
(viii) ABT-963 (described in International Patent Application Publication No. WO 00/24719)
(ix) Nimesulide (described in U.S. Pat. No. 3,840,597), flosulide (discussed in J. Carter, Exp. Opin. Ther. Patents, 8(1), 21-29 (1997)), NS-398 (disclosed in U.S. Pat. No. 4,885,367), SD 8381 (described in U.S. Pat. No. 6,034,256), BMS-347070 (described in U.S. Pat. No. 6,180,651), S-2474 (described in European Patent Publication No. 595546) and MK-966 (described in U.S. Pat. No. 5,968,974);
(x) The compounds and pharmaceutically acceptable derivatives described in U.S. Pat. Nos. 6,395,724, 6,077,868, 5,994,381, 6,362,209, 6,080,876, 6,133,292, 6,369,275, 6,127,545, 6,130,334, 6,204,387, 6,071,936, 6,001,843, 6,040,450, International Patent Application Publication No WO 96/03392, International Patent Application Publication No WO 96/24585, U.S. Pat. Nos. 6,340,694, 6,376,519, 6,153,787, 6,046,217, 6,329,421, 6,239,137, 6,136,831, 6,297,282, 6,239,173, 6,303,628, 6,310,079, 6,300,363, 6,077,869, 6,140,515, 5,994,379, 6,028,202, 6,040,320, 6,083,969, 6,306,890, 6,307,047, 6,004,948, 6,169,188, 6,020,343, 5,981,576, 6,222,048, 6,057,319, 6,046,236, 6,002,014, 5,945,539, 6,359,182, International Patent Application Publication No. WO 97/13755, International Patent Application Publication No. WO 96/25928, International Patent Application Publication No. WO 96/374679, International Patent Application Publication No. WO 95/15316, International Patent Application Publication No. WO 95/15315, International Patent Application Publication No. WO 96/03385, International Patent Application No. WO 95/00501, International Patent Application No. WO 94/15932, International Patent Application Publication No. WO 95/00501, International Patent Application Publication No. WO 94/27980, International Patent Application Publication No. WO 96/25405, International Patent Application Publication No. WO 96/03388, International Patent Application Publication No. WO 96/03387, U.S. Pat. No. 5,344,991, International Patent Application Publication No. WO 95/00501, International Patent Application Publication No. WO 96/16934, International Patent Application Publication No. WO 96/03392, International Patent Application Publication No. WO 96/09304, International Patent Application Publication No. WO 98/47890, and International Patent Application Publication No. WO 00/24719.

The contents of any of the patent applications, and in particular the general formulae of the therapeutically active compounds of the claims and exemplified compounds therein, are incorporated herein in their entirety by reference thereto.

Generally, the compounds of the present invention will be administered as a formulation in association with one or more pharmaceutically acceptable excipients. The term “excipient” is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in “Remington's Pharmaceutical Sciences”, 19^{th} Edition (Mack Publishing Company, 1995).

Thus, according to another aspect of the present invention, there is provided a pharmaceutical formulation comprising a compound of formula (I) in admixture with a pharmaceutically acceptable adjuvant, diluent or carrier.

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids or powders, lozenges (including liquid-filled), chews, multi- and nano-particulates, gels, solid solution, liposome, films (including muco-adhesive), ovules, sprays and liquid formulations.

Liquid formulations include suspensions, solutions, syrups and elixirs. Such formulations may be employed as fillers in soft or hard capsules and typically comprise a carrier, for example water, ethanol, polyethylene glycol, propylene glycol, methylcellulose, or a suitable oil, and one or more emulsifying agents and/or suspending agents. Liquid formulations may also be prepared by the reconstitution of a solid, for example from a sachet.

The compounds of the invention may also be used in fast-dissolving, fast disintegrating dosage forms such as those described in Expert Opinion in Therapeutic Patents, 11 (6), 981-986 by Liang and Chen (2001).

For tablet dosage forms, depending on dose, the drug may make up from 1 wt % to 80 wt % of the dosage form, more typically from 5 wt % to 60 wt % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 wt % to 25 wt %, preferably from 5 wt % to 20 wt %, of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 wt % to 5 wt % of the tablet, and glidants may comprise from 0.2 wt % and 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 wt % to 10 wt %, preferably from 0.5 wt % to 3 wt %, of the tablet.

Other possible ingredients include anti-oxidants, colourants, flavouring agents, preservatives and taste-masking agents.

Exemplary tablets contain up to about 80% drug, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in “Pharmaceutical Dosage Forms: Tablets, Vol. 1”, by H. Lieberman and L. Lachman, Marcel Dekker, N.Y., N.Y., 1980 (ISBN 0-8247-6918-X).

Solid formulations for oral administration may be formulated to be immediate and/or modified release. Modified release formulations include delayed-, sustained-, pulsed-, controlled-, targeted- and programmed release.

Suitable modified release formulations for the purposes of the invention are described in U.S. Pat. No. 6,106,864. Details of other suitable release technologies such as high energy dispersions and osmotic and coated particles are to be found in Verma et al, Pharmaceutical Technology On-line, 25(2), 1-14 (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intreperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by suitable processing, for example, the use of high energy spray-dried dispersions (see WO 01/47495) and/or by the use of appropriate formulation techniques, such as the use of solubility-enhancing agents.

Formulations for parenteral administration may be formulated to be immediate and/or modified release. Modified release formulations include delayed-, sustained-, pulsed-, controlled-, targeted- and programmed release. Thus, compounds of the invention may be formulated as a solid, semi-solid, or thixotropic liquid for administration as an implanted depot providing modified release of the active compound. Examples of such formulations include drug-coated stents and PGLA microspheres.

The compounds of the invention may also be administered topically to the skin or mucosa, either dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J. Pharm. Sci., 88 (10), 955-958 by Finnin and Morgan (October 1999).

Other means of topical administration include delivery by iontophoresis, electroporation, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer or nebuliser contains a solution or suspension of the compound(s) of the invention comprising, for example, ethanol, aqueous ethanol, or a suitable alternative agent for dispersing, solubilising or extending release of the active, the propellant(s) as solvent and an optional surfactant, such as sorbitan trioleate, oleic acid, or an oligolactic acid.

Prior to use in a dry powder of suspension formulation, the drug product is micronised to a size suitable for delivery by inhalation (typically less than 5 microns). This may be achieved by any appropriate comminuting method, such a spiral jet milling, fluid bed jet milling, supercritical fluid processing to form nanoparticles, high pressure homogenisation or spray drying.

Capsules (made, for example, from gelatin or HPMC), blisters and cartridges for use in an inhaler or insufflator may be formulated to contain a powder mix of the compound of the invention, a suitable powder base such as lactose or starch and a performance modifier such as /-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula (I), propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, poly-DL-lactic-coglycolic acid (PGLA). Modified release formulations include delayed-, sustained-, pulsed-, controlled-, targeted and programmed release.

The compounds of the invention may be administered rectally or vaginally, for example, in the form of a suppository, pessary or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

The compounds of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

The compounds of the invention may be combined with soluble macromolecular entities such as cyclodextrin or polyethylene glycol-containing polymers to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent or solubiliser. Most commonly used for these purposes are alpha-, beta- and gamma-cyclodextrins, examples of which may be found in International Patent Applications Nos. WO 91/11172, WO 94/02518 and WO 98/55148.

Inasmuch as it may be desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions.

Thus the kit of the invention comprises two or more separate pharmaceutical compositions, at least one of which contains a compounds of formula (I) in accordance with the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

For administration to human patients, the total daily dose of the compounds of the invention will typically be in the range of from about 0.01 to about 15 mg/kg of body weight, depending on the mode of administration. The total daily dose may be administered in a single dose or divided doses throughout the day. These dosages are based on an average human subject having a weight of about 65 kg to 70 kg. The physician will readily be able to determine doses for subjects whose weight falls outside this range, such as infants and the elderly.

As used herein, the terms “treating” and “to treat”, mean to alleviate symptoms, eliminate the causation either on a temporary or permanent basis, or to prevent or slow the appearance of symptoms. The term “treatment” includes alleviation, elimination of causation (either on a temporary or permanent basis) of, or prevention of symptoms and disorders associated with primary and/or secondary dysmenorrhoea. The treatment may be a pre-treatment as well as a treatment at the on-set of symptoms.

The compounds of the present invention may be tested in the screens set out below:

1.0 V_{1A} Filter Binding Assay

1.1 Membrane Preparation

Receptor binding assays were performed on cellular membranes prepared from CHO cells stably expressing the human V_{1A} receptor, (CHO-hV_{1A}). The CHO-hV_{1A} cell line was kindly provided under a licensing agreement by Marc Thibonnier, Dept. of Medicine, Case Western Reserve University School of Medicine, Cleveland, Ohio. CHO-hV_{1A} cells were routinely maintained at 37° C. in humidified atmosphere with 5% CO₂ in DMEM/Hams F12 nutrient mix supplemented with 10% fetal bovine serum, 2 mM L-glutamine, 15 mM HEPES and 400 μg/ml G418. For bulk production of cell pellets, adherent CHO-hV_{1A} cells were grown to confluency of 90-100% in 850 cm² roller bottles containing a medium of DMEM/Hams F12 Nutrient Mix supplemented with 10% fetal bovine serum, 2 mM L-glutamine and 15 mM HEPES. Confluent CHO-hV_{1A} cells were washed with phosphate-buffered saline (PBS), harvested into ice cold PBS and centrifuged at 1,000 rpm. Cell pellets were stored at −80° C. until use. Cell pellets were thawed on ice and homogenised in membrane preparation buffer consisting of 50 mM Tris-HCl, pH 7.4, 5 mM MgCl₂ and supplemented with a protease inhibitor cocktail, (Roche). The cell homogenate was centrifuged at 1000 rpm, 10 min, 4° C. and the supernatant was removed and stored on ice. The remaining pellet was homogenised and centrifuged as before. The supernatants were pooled and centrifuged at 25,000×g for 30 min at 4° C. The pellet was resuspended in freezing buffer consisting of 50 mM Tris-HCl, pH 7.4, 5 mM MgCl₂ and 20% glycerol and stored in small aliquots at −80° C. until use. Protein concentration was determined using Bradford reagent and BSA as a standard.

1.2 V_{1A} Filter Binding

Protein linearity followed by saturation binding studies were performed on each new batch of membrane. Membrane concentration was chosen that gave specific binding on the linear portion of the curve. Saturation binding studies were then performed using various concentrations of [³H]-arginine vasopressin, [³H]-AVP (0.05 nM-100 nM) and the K_{d} and Bₘₐₓ determined.

Compounds were tested for their effects on [³H]-AVP binding to CHO-hV_{1A} membranes, (³H-AVP; specific activity 65.5 Ci/mmol; NEN Life Sciences). Compounds were solubilised in dimethylsulfoxide (DMSO) and diluted to working concentration of 10% DMSO with assay buffer containing 50 mM Tris-HCL pH 7.4, 5 mM MgCl₂ and 0.05% BSA. 25 μl compound and 25 μl [³H]-AVP, (final concentration at or below K_{d} determined for membrane batch, typically 0.5 nM-0.6 nM) were added to a 96-well round bottom polypropylene plate. The binding reaction was initiated by the addition of 200 μl membrane and the plates were gently shaken for 60 min at room temperature. The reaction was terminated by rapid filtration using a Filtermate Cell Harvester (Packard Instruments) through a 96-well GF/B UniFilter Plate which had been presoaked in 0.5% polyethyleneimine to prevent peptide sticking. The filters were washed three times with 1 ml ice cold wash buffer containing 50 mM Tris-HCL pH 7.4 and 5 mM MgCl₂. The plates were dried and 50 μl Microscint-0 (Packard instruments) was added to each well. The plates were sealed and counted on a TopCount Microplate Scintillation Counter (Packard Instruments). Non-specific binding (NSB) was determined using 1 μM unlabelled d(CH2)5Tyr(Me)AVP ([β-mercapto-β,β-cyclopentamethylenepropionyl,0-Me-Tyr²,Arg⁸]-vasopressin) (βMCPVP), (Sigma). The radioligand binding data was analysed using a four parameter logistic equation with the min forced to 0%. The slope was free fitted and fell between −0.75 and −1.25 for valid curves. Specific binding was calculated by subtracting the mean NSB cpm from the mean Total cpm. For test compounds the amount of ligand bound to the receptor was expressed as % bound=(sample cpm−mean NSB cpm)/specific binding cpm×100. The % bound was plotted against the concentration of test compound and a sigmoidal curve was fitted. The inhibitory dissociation constant (Kᵢ) was calculated using the Cheng-Prusoff equation: Kᵢ=IC₅₀/(1+[L]/K_{d}) where [L] is the concentration of ligand present in the well and K_{d} is the dissociation constant of the radioligand obtained from Scatchard plot analysis.

2.0 V_{1A} Functional Assay: Inhibition of AVP/V_{1A}-R Mediated Ca²⁺ Mobilization by FLIPR (Fluorescent Imaging Plate Reader) (Molecular Devices)

Intracellular calcium release was measured in CHO-hV_{1A} cells using FLIPR, which allows the rapid detection of calcium following receptor activation. The CHO-hV_{1A} cell line was kindly provided under a licensing agreement by Marc Thibonnier, Dept. of Medicine, Case Western Reserve University School of Medicine, Cleveland, Ohio. CHO-V_{1A} cells were routinely maintained at 37° C. in humidified atmosphere with 5% CO₂ in DMEM/Hams F12 nutrient mix supplemented with 10% fetal bovine serum, 2 mM L-glutamine, 15 mM HEPES and 400 μg/ml G418. On the afternoon before the assay cells were plated at a density of 20,000 cells per well into black sterile 96-well plates with clear bottoms to allow cell inspection and fluorescence measurements from the bottom of each well. Wash buffer containing Dulbecco's phosphate buffered saline (DPBS) and 2.5 mM probenecid and loading dye consisting of cell culture medium containing 4 μM Fluo-3-AM (dissolved in DMSO and pluronic acid), (Molecular Probes) and 2.5 mM probenecid was prepared fresh on the day of assay. Compounds were solubilised in DMSO and diluted in assay buffer consisting of DPBS containing 1% DMSO, 0.1% BSA and 2.5 mM probenecid. The cells were incubated with 100 μl loading dye per well for 1 hour at 37° C. in humidified atmosphere with 5% CO₂. After dye loading the cells were washed three times in 100 μl wash buffer using a Denley plate washer. 100 μl wash buffer was left in each well. Intracellular fluorescence was measured using FLIPR. Fluorescence readings were obtained at 2 s intervals with 50 μl of the test compound added after 30 s. An additional 155 measurements at 2 s intervals were then taken to detect any compound agonistic activity. 50 μl of arginine vasopressin (AVP) was then added so that the final assay volume was 200 μl. Further fluorescence readings were collected at 1 s intervals for 120 s. Responses were measured as peak fluorescence intensity (FI). For pharmacological characterization a basal FI was subtracted from each fluorescence response. For AVP dose response curves, each response was expressed as a % of the response to the highest concentration of AVP in that row. For IC₅₀ determinations, each response was expressed as a % of the response to AVP. IC50 values were converted to a modified K_{b} value using the Cheng-Prusoff equation which takes into account the agonist concentration, [A], the agonist EC₅₀ and the slope: K_{b}=IC₅₀/(2+[A]/A₅₀]ⁿ)^{1/n}−1 where [A] is the concentration of AVP, A₅₀ is the EC₅₀ of AVP from the dose response curve and n=slope of the AVP dose response curve.

The compounds of the invention may have the advantage that they are more potent, have a longer duration of action, have a broader range of activity, are more stable, have fewer side effects or are more selective, or have other more useful properties than the compounds of the prior art.

The invention is illustrated by the following preparations and examples:

### Preparation 1: 4-[N′-(2-Chloro-acetyl)-hydrazinocarbonyl]-piperidine-1-carboxylic acid tert-butyl ester (II)

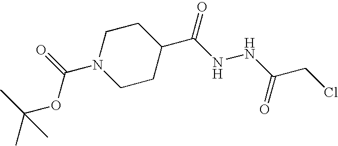

4-Hydrazinocarbonyl-piperidine-1-carboxylic acid tert-butyl ester (see reference WO 9703986 A1 19970206) (25 g, 103 mmol) was dissolved in dichloromethane (300 ml) and 4-methylmorpholine (12.5 ml, 113 mmol) was added. The mixture was cooled using an ice bath and chloroacetyl chloride (8.2 ml, 103 mmol) was added drop wise. The reaction was warmed to room temperature and was stirred for 4 hours. The reaction mixture was partitioned with aqueous sodium hydrogen carbonate solution, dried over magnesium sulphate, filtered and the filtrate evaporated to give the title compound as an off white solid (29.6 g).

Found; C, 48.01; H, 6.91; N, 12.85; C₁₃H₂₂N₃O₄Cl 0.3 H₂O requires; C, 48.02; H, 7.01; N, 12.92%; APCI MS m/z 318 [M−H]⁺.

### Preparation 2: 4-(5-Chloromethyl-[1,3,4]oxadiazol-2-yl)-piperidine-1-carboxylic acid tert-butyl ester (III)

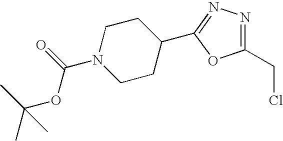

Hydrazide (II) (5.0 g, 15.6 mmol) was suspended in dichloromethane (200 ml) and pyridine (6.4 ml, 78 mmol) was added before cooling the mixture to 10° C. Trifluoroacetic anhydride (6.6 ml, 39 mmol) was added drop wise over 15 minutes and then stirred at room temperature for 3 hours. The reaction mixture was partitioned with water (50 ml), the organic layer was dried over magnesium sulphate, then filtered and the filtrate was evaporated under reduced pressure. The residue was purified by chromatography on silica gel using methanol in dichloromethane as eluant (2:98) to afford the title compound as a white solid (2.95 g).

¹H NMR (400 MHz, CD₃OD): δ 1.45 (s, 9H), 1.74 (m, 2H), 2.19 (m, 2H), 3.04 (m, 2H), 3.24 (m, 1H), 4.09 (m, 2H), 4.85 (s, 2H)

### Preparation 3: (2-Amino-5-methoxy-phenyl)-methanol (IV)

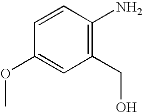

2-Amino-5-methoxy-benzoic acid (2.0 g, 12 mmol) in tetrahydrofuran (20 ml) was added drop wise to an ice cooled 1 molar solution of lithium aluminium hydride (14.4 ml) in tetrahydrofuran and stirred at 5° C. for 2 hours. Water (0.5 ml) was added drop wise, followed by a 2 molar aqueous sodium hydroxide solution (0.5 ml). The resulting emulsion was dried over magnesium sulphate, filtered and then evaporated under reduced pressure to afford the title compound as a yellow solid (766 mg).

¹H NMR (400 MHz, CD₃OD): δ 3.70 (s, 3H), 4.55 (s, 2H), 6.65-6.78 (m, 3H); APCI MS m/z 154 [M+H]⁺

### Preparation 4: 4-[5-(2-Amino-5-chloro-benzyloxymethyl)-[1,3,4]oxadiazol-2-yl]-piperidine-1-carboxylic acid tert-butyl ester (V)

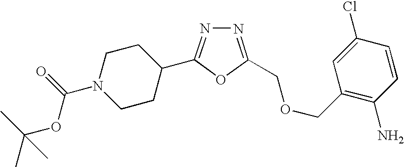

A solution of (2-Amino-5-chloro-phenyl)-methanol (1 g, 6.4 mmol) in tetrahydrofuran (10 ml) was added drop wise to an ice cooled suspension of sodium hydride (60% in mineral oil, 215 mg, 5.4 mmol) in tetrahydrofuran (5 ml) and stirred for 1 hour. A solution of oxadiazole (III) (1 g, 5.3 mmol) in tetrahydrofuran (5 ml) was added drop wise and the mixture was stirred at room temperature for 2 hours. The reaction mixture was partitioned between dichloromethane (50 ml) and sodium hydrogen carbonate solution (25 ml). The aqueous solution was washed with dichloromethane (2×20 ml) and the combined organic layers were dried over magnesium sulphate and evaporated under reduced pressure. The residue was purified by chromatography on silica gel using methanol in dichloromethane (5:95) as eluant to give the title compound (1.3 g) as a yellow solid.

¹H NMR (400 MHz, CDCl₃): δ 1.47 (s, 9H), 1.81 (m, 2H), 2.07 (m, 2H), 2.96 (m, 2H), 3.08 (m, 1H), 4.12 (m, 2H), 4.23 (s, 2H), 4.58 (s, 2H), 4.68 (s, 2H), 6.62 (d, 1H), 7.07 (s, 1H), 7.12 (d, 1H); APCI MS m/z 423 [MH]⁺, 323 [M-Boc]⁺

### Preparation 5: 4-(8-Chloro-4H,6H-5-oxa-2,3,10b-triaza-benzo[e]azulen-1-yl)-piperidine-1-carboxylic acid tert-butyl ester (VI)

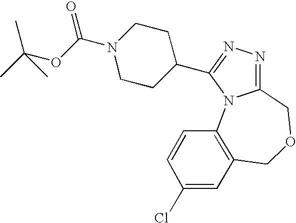

Toluene-4-sulfonic acid (80 mg, 0.46 mmol) was added to a solution of oxadiazole (V) (1.28 g, 3.0 mmol) in xylene and heated to 140° C. for 18 hours. The xylene was removed under reduced pressure and the residue was partitioned between dichloromethane (100 ml) and sodium hydrogen carbonate solution (25 ml). The aqueous solution was washed with dichloromethane (2×20 ml) and the combined organic layers were dried over magnesium sulphate and evaporated under reduced pressure. The residue was purified by chromatography on silica gel using methanol and ammonium hydroxide in dichloromethane (5:0.5:95) as eluant to give the title compound (730 mg) as a pale yellow foam.

¹H NMR (400 MHz, CDCl₃): δ 1.43 (s, 9H), 1.85 (m, 2H), 1.96 (m, 2H), 2.92 (m, 2H), 3.08 (m, 1H), 4.18 (m, 2H), 4.40 (s, 2H), 4.66 (s, 2H), 7.36 (d, 1H), 7.58 (m, 2H); Found; C, 57.98; H, 6.17; N, 13.40; C₂₀H₂₅N₄O₃Cl 0.5H₂O requires; C, 58.04; H, 6.33; N, 13.54%; APCI MS m/z 405 [MH]⁺, 305 [M-Boc]⁺.

### Preparation 6: 8-Chloro-1-piperidin-4-yl-4H,6H-5-oxa-2,3,10b-triaza-benzo[e]azulene (VII′)

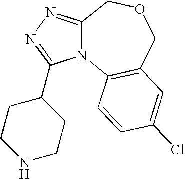

Triazole (VI) (700 mg, 1.73 mmol) was dissolved in 1,4-dioxan (6 ml) and hydrochloric acid (4M in 1,4-dioxane, 12 ml) was added. The reaction mixture was stirred at room temperature for 4 hours. The 1,4-dioxane was removed under reduced pressure and the residue was partitioned between dichloromethane (100 ml) and sodium hydrogen carbonate solution (25 ml). The aqueous solution was washed with dichloromethane (2×20 ml) and the combined organic layers were dried over magnesium sulphate and evaporated under reduced pressure to give the title compound (410 mg) as a pale yellow foam.

¹H NMR (400 MHz, CD₃OD): δ 1.83 (m, 4H), 2.65 (t, 2H), 3.09 (m, 2H), 3.24 (m, 1H), 4.41 (s, 2H), 4.58 (s, 2H), 7.58 (m, 3H); APCI MS m/z 305 [MH]⁺

### Preparation 7: (5-Chloro-2-nitro-benzyl)-methyl-amine (IX):

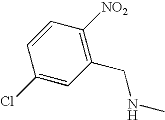

To a solution of 15.0 g of 5-chloro-2-nitrobenzaldehyde (VIII) (81 mmoles, 1 eq.) in 400 ml of methylene chloride were added 33.8 ml of triethylamine (243 mmoles, 3 eq.) and 16.4 g of methylamine hydrochloride (243 mmoles, 3 eq.). The reaction mixture was stirred for 16 hours. An aqueous solution of sodium bicarbonate was added, the methylene chloride phase dried (MgSO₄), and the volatiles were removed under reduced pressure. The residue was dissolved in methanol, the solution cooled to 0° C., and 2.4 g of sodium borohydride (65 mmoles, 0.8 eq.) added portionwise. The solution was stirred for 4 hours, the solvent removed under reduced pressure and the residue partitioned between methylene chloride and an aqueous solution of sodium bicarbonate. The organic phase was dried over magnesium sulfate and filtered. The volatiles were removed under reduced pressure affording 14.8 g of the title compound as a brown solid (91%).

¹H NMR (400 MHz, CDCl₃): δ 2.45 (s, 3H), 4.00 (s, 2H), 7.40 (d, 1H,), 7.65 (s, 1H), 7.95 (d, 1H); LCMS: m/z APCI⁺, 201 [MH]⁺

### Preparation 8: 4-Chloro-2-methylaminomethyl-phenylamine (X)

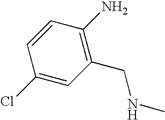

To a solution of 14.6 g of compound (IX) (73 mmoles, 1 eq.) in 350 ml of ethanol was added 500 mg of PtO₂. The mixture was stirred under 40 PSI of hydrogen for 2 hours, filtered over Celite® and the volatiles were then removed under reduced pressure, affording 12.1 g of a green oil. This oil was purified by column chromatography on silica gel using methylene chloride/methanol/aqueous ammonia as eluant (90:10:1 v/v/v to 95:5 v/v) to afford 11.40 g of the title compound as an oil (92%).

¹H NMR (400 MHz, CDCl₃): δ 2.40 (s, 3H), 3.70 (s, 2H), 4.65 (m, 2H), 6.60 (d, 1H,), 7.00 (s, 1H), 7.05 (d, 1H); LCMS: m/z APCI⁺, 171 [MH]⁺

### Preparation 9: 4-(5-{[(2-Amino-5-chloro-benzyl)-methyl-amino]-methyl}-[1,3,4]oxadiazol-2-yl)-piperidine-1-carboxylic acid tert-butyl ester (XI):

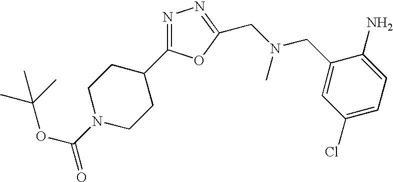

To a solution of 10.0 g of oxadiazole (III) (33 mmoles, 1 eq.) in 220 ml of THF was added 6.8 g of phenylamine (X) (40 mmoles, 1.2 eq.) and 6.9 ml of triethylamine (50 mmoles, 1.5 eq.). The solution was refluxed for 24 hours, the solvent removed under reduced pressure and the residue partitioned between methylene chloride and an aqueous solution of sodium bicarbonate. The organic phase was separated and dried over magnesium sulfate and filtered. The volatiles were removed under reduced pressure and the residue was purified by column chromatography on silica gel using methylene chloride/methanol/aqueous ammonia as eluant (97:3:0.3 v/v/v to 95:5:5 v/v/v), to afford 10.7 g of the title compound (74.1%).

¹H NMR (400 MHz, CDCl₃): δ 1.40 (s, 9H), 1.80 (m, 2H), 2.00 (m, 2H), 2.25 (s, 3H), 2.90 (m, 2H), 3.05 (m, 1H), 3.60 (s, 2H), 3.80 (s, 2H), 4.05 (m, 2H), 6.60 (d, 1H,), 7.00 (s, 1H), 7.05 (d, 1H); LCMS: m/z APCI⁺, 436 [MH]⁺

### Preparation 10: 8-Chloro-5-methyl-1-piperidin-4-yl-5,6-dihydro-4H-2,3,5,10b-tetraaza-benzo[e]azulene (VII″)

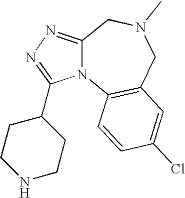

A solution of 10.7 g of ester (XI) (25 mmoles, 1 eq.) in 200 ml of toluene was heated at 50° C., and 2.84 ml of TFA (38 mmoles, 1.5 eq.) were added. The solution was refluxed for 1 hour and the solvent was removed under reduced pressure. The residue was partitioned between methylene chloride and an aqueous solution of sodium hydroxide. The aqueous phase was concentrated under reduced pressure and purified by column chromatography on silica gel using methylene chloride/methanol/aqueous ammonia as eluant (80:20:2 v/v/v to 90:10:1 v/v/v), to afford 4.6 g of the title compound (59%)

¹H NMR (400 MHz, CD₃OD): δ 1.80-2.20 (m, 4H), 2.50 (s, 3H), 2.60-2.90 (m, 2H), 3.00 (m, 2H), 3.40 (m, 3H), 3.40-3.80 (m, 2H), 7.60 (m, 3H); LCMS: m/z APCI⁺, 318 [MH]⁺

### EXAMPLES 1 to 92

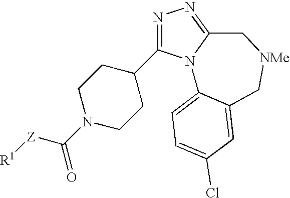

Examples 1 to 92, illustrated in Table 1, were synthesised as a library from intermediates of formula (VII). The following monomer solutions were used:

| | |
|---|---|
| Carboxylic | Dissolved in dimethylacetamide (DMA) (anhydrous) plus |
| Acids: | 3.75% triethylamine at 0.2M concentration |
| Amines: | Dissolved in DMA (anhydrous) plus 3.75% triethylamine at |
| | 0.2M concentration |
| Amines | Dissolved in DMA (anhydrous) + 3.75% triethylamine at |
| as Salts: | 0.2M concentration |
| HBTU: | Dissolved in DMA (anhydrous) at 0.2M concentration |

N.B. Gentle sonication, in a warm water bath (temp <40° C.), was used to dissolve the monomers where necessary.

Experimental Procedure:

The reaction Scale was between 20 and 30 micromoles per well (experimental details shown for 20 μmole reaction, scale can be adjusted accordingly within this range). Reactions were performed in a polypropylene 96 well plate.
a) Amine solutions (0.1 ml, 20 μmoles, 1 eq.) were added to the wells
b) Carboxylic acid solutions (0.15 ml, 30 μmoles, 1.5 eq.) were added to the wells
c) HBTU Solution (0.15 ml, 30 μmoles, 1.5 eq.) was added to each well
d) The polypropylene 96 well plate was sealed with a PTFE and rubber gasket and clamped between a pair of metal plates.
e) The plate was heated in an oven for 6 hours at 60° C. and then left to cool down in the oven overnight.
f) When cool, the plate was unclamped and placed in a Genevac to remove the solvent.
g) The samples were re-dissolved in DMSO/water (9:1) (500 μl) and any particulate matter was removed by filtration.
h) Purification was carried out by RP-HPLC.

HPLC Purification Conditions:
Column: Phenomenex Luna C18, 10 um, 150×10 mm id
Temperature: ambient
Eluent A: 0.05% Diethylamine in water
Eluent B: Acetonitrile
Samples dissolved in: 90% Dimethylsulphoxide in water.
Sample loaded using Gilson Autosampler with Injection Volume of 550 μl

Gilson LC Pump Initial Conditions:

Solvents

| | |
|---|---|
| A % | 80.0 |
| B % | 20.0 |
| Flow (ml/min) | 8.000 |

Gilson LC Pump Gradient Timetable:

| Time | A % | B % | Flow (ml/min) |
|---|---|---|---|
| 0.00 | 80.0 | 20.0 | 8.000 |
| 0.20 | 80.0 | 20.0 | 8.000 |
| 7.00 | 5.0 | 95.0 | 8.000 |
| 9.00 | 5.0 | 95.0 | 8.000 |
| 9.10 | 80.0 | 20.0 | 8.000 |
| 10.50 | 80.0 | 20.0 | 8.000 |

Gilson 119 uv Detector Monitoring at 254 nm:
Collector set at 225 nm
Dual sensitivity 200
Peak sensitivity 80
Peak width 0.3 min.

HPLC Analysis Conditions and Mass Spectrometer Details:
Column: Phenomenex Luna C18, 5 um, 30×4.6 mm id.
Eluent A: 0.05% Diethylamine in water
Eluent B: Acetonitrile
Samples dissolved in: 90% Dimethylsulphoxide in water
Sample loaded using Gilson Quad Z with Injection Volume of 5 μl

Waters 1525 Binary LC Pump Initial Conditions:

Solvents

| | |
|---|---|
| A % | 95.0 |
| B % | 5.0 |
| Flow (ml/min) | 2.5 (per channel) |
| Temperature (° C.) | ambient |

LC Pump Gradient Timetable:

The gradient Timetable contains 4 entries which are:

| Time | A % | B % | Flow |
|---|---|---|---|
| 0.00 | 95.0 | 5.0 | 2.500 |
| 3.00 | 5.0 | 95.0 | 2.500 |
| 3.50 | 95.0 | 5.0 | 2.500 |

Total run time 4.50 mins

Detection:

Waters 2488 dual wavelength detector

| | |
|---|---|
| UV1 (nm) | 225 |
| UV2 (nm) | 255 |

Mass Spectrometer:

Waters ZQ 2000 4 way MUX,

| | |
|---|---|
| ES+ Cone voltage: 26 v | Capillary: 3.85 kv |
| ES− Cone voltage: −30 v | Capillary: −3.00 kv |
| Desolvation gas: 800 l/min | |
| Source Temp: 300° C. | |
| Scan range 160-1000 Da | |

TABLE 1

| | | | | HPLC |
|---|---|---|---|---|
| E.g. | | | Mass Ion | Retⁿ time |
| No | Z | R¹ | found | (mins) |
| 1 | — | 1H-indol-3-yl | 461.18 | 1.55 |
| 2 | CH₂ | o-tolyl | 450.20 | 1.6 |
| 3 | CHCH₂ | phenyl | 450.20 | 1.72 |
| 4 | — | o-tolyl | 436.18 | 1.6 |
| 5 | — | 4-methoxy-phenyl | 452.18 | 1.47 |
| 6 | — | n-butyl | 402.20 | 1.55 |
| 7 | CH₂ | cyclopentyl | 428.21 | 1.49 |
| 8 | — | iso-propyl | 402.20 | 1.52 |
| 9 | — | 1-methyl-cyclohexyl | 442.23 | 1.85 |
| 10 | CH₂ | 2-methoxy-phenyl | 466.19 | 1.64 |
| 11 | — | 1-methyl-propyl | 402.20 | 1.42 |
| 12 | CHCH₂ | phenoxyl | 466.19 | 1.42 |
| 13 | CH₂ | 2-fluoro-phenyl | 454.17 | 1.65 |
| 14 | — | 2-methyl-butyl | 416.21 | 1.65 |
| 15 | CH₂ | 3-fluoro-phenyl | 454.17 | 1.57 |
| 16 | CH₂ | m-tolyl | 450.20 | 1.72 |
| 17 | CH₂ | p-tolyl | 450.20 | 1.64 |
| 18 | CH₂ | cyclopropyl | 400.18 | 1.42 |
| 19 | — | 3-methyl-butyl | 416.21 | 1.65 |
| 20 | — | tetrahydro-furan-2-yl | 416.18 | 1.18 |
| 21 | CH₂ | o-tolyloxy | 466.19 | 1.42 |
| 22 | CH₂ | t-butyl | 416.21 | 1.55 |
| 23 | — | cyclopentyl | 414.20 | 1.57 |
| 24 | CH₂ | 4-fluoro-phenyl | 454.17 | 1.57 |
| 25 | — | n-pentyl | 416.21 | 1.68 |
| 26 | CH₂ | 3-methoxy-phenyl | 466.19 | 1.62 |
| 27 | C₃H₆ | phenyl | 464.21 | 1.8 |
| 28 | CH₂ | phenyl | 436.18 | 1.6 |
| 29 | — | 4-methyl-pentyl | 430.23 | 1.85 |
| 30 | CH₂ | 3,5-dimethyl-phenyl | 464.21 | 1.84 |
| 31 | — | phenyl | 422.17 | 1.47 |
| 32 | — | 2-chloro-phenyl | 456.13 | 1.62 |
| 33 | — | 4-chloro-phenyl | 456.13 | 1.64 |
| 34 | — | 4-ethoxy-phenyl | 466.19 | 1.67 |
| 35 | — | 4-methylsulfanyl-phenyl | 468.15 | 1.72 |
| 36 | — | 2-ethoxy-phenyl | 466.19 | 1.35 |
| 37 | — | 2,6-difluoro-phenyl | 458.15 | 1.59 |
| 38 | — | 4-ethyl-phenyl | 450.20 | 1.8 |
| 39 | — | 2,5-difluoro-phenyl | 458.15 | 1.62 |
| 40 | — | 2,3-difluoro-phenyl | 458.15 | 1.57 |
| 41 | — | 2,4-difluoro-phenyl | 458.15 | 1.6 |
| 42 | — | 3,4-difluoro-phenyl | 458.15 | 1.67 |
| 43 | — | 4-isopropyl-phenyl | 464.21 | 1.92 |
| 44 | — | 6-methyl-pyridin-3-yl | 437.18 | 1.24 |
| 45 | — | 3,5-difluoro-phenyl | 458.15 | 1.65 |
| 46 | — | 1H-benzoimidazol-5-yl | 462.17 | 1.24 |
| 47 | — | 3-fluoro-2-methyl-phenyl | 454.17 | 1.59 |
| 48 | — | 2-fluoro-5-methyl-phenyl | 454.17 | 1.7 |
| 49 | — | 2-fluoro-phenyl | 440.16 | 1.55 |
| 50 | — | 3-chloro-phenyl | 456.13 | 1.62 |
| 51 | — | 4-benzonitrile | 447.16 | 1.5 |
| 52 | — | 3-fluoro-4-methyl-phenyl | 454.17 | 1.72 |
| 53 | — | 3-methoxy-4-methyl-phenyl | 466.19 | 1.65 |
| 54 | — | 6-methyl-pyridin-2-yl | 437.18 | 1.34 |
| 55 | — | m-tolyl | 436.18 | 1.67 |
| 56 | — | p-tolyl | 436.18 | 1.65 |
| 57 | — | 3-ethoxy-phenyl | 466.19 | 1.62 |
| 58 | — | 2-ethyl-5-methyl-2H-pyrazol-3-yl | 454.20 | 1.42 |
| 59 | — | 2,3-dihydro-benzofuran-7-yl | 464.18 | 1.57 |
| 60 | — | 4-methoxy-3-methyl-phenyl | 466.19 | 1.62 |
| 61 | — | benzo[1,2,5]oxadiazol-5-yl- | 464.15 | 1.62 |
| 62 | CH₂ | 2,4-dimethyl-phenyl | 464.21 | 1.82 |
| 63 | — | 5-isopropyl-2-methyl-2H-pyrazol-3-yl | 468.22 | 1.65 |
| 64 | — | 1H-indol-2-yl | 461.18 | 1.75 |
| 65 | — | 2,3-dihydro-benzofuran-5-yl | 464.18 | 1.47 |
| 66 | — | 2-hydroxy-5-methyl-phenyl | 452.18 | 1.52 |
| 67 | — | 1H-indol-6-yl | 461.18 | 1.52 |
| 68 | — | 1H-indol-5-yl | 461.18 | 1.52 |
| 69 | — | 2-methyl-5-propyl-2H-pyrazol-3-yl | 468.22 | 1.5 |
| 70 | — | 3-methoxy-4-methyl-phenyl | 466.19 | 1.65 |
| 71 | — | 6-methyl-pyridin-2-yl | 437.18 | 1.34 |
| 72 | — | m-tolyl | 436.18 | 1.67 |
| 73 | — | p-tolyl | 436.18 | 1.65 |
| 74 | — | 3-ethoxy-phenyl | 466.19 | 1.62 |
| 75 | — | 4-methyl-pyridin-3-yl | 437.18 | 1.27 |
| 76 | — | 2-ethyl-5-methyl-2H-pyrazol-3-yl | 454.20 | 1.42 |
| 77 | — | 2,3-dihydro-benzofuran-7-yl | 464.18 | 1.57 |
| 78 | — | 4-methoxy-3-methyl-phenyl | 466.19 | 1.62 |
| 79 | — | 2-ethoxy-pyridin-3-yl | 467.19 | 1.47 |
| 80 | — | Benzo[1,2,5]oxadiazol-5-yl | 464.15 | 1.62 |
| 81 | C₂H₆ | m-tolyl | 464.21 | 1.8 |
| 82 | CH₂ | 2,4-dimethyl-phenyl | 464.21 | 1.82 |
| 83 | CH₂ | piperidin-1-yl | 443.22 | 1.1 |
| 84 | — | 5-isopropyl-2-methyl-2H-pyrazol-3-yl | 468.22 | 1.65 |
| 85 | — | 1H-indol-2-yl | 461.18 | 1.75 |
| 86 | — | 2,3-dihydro-benzofuran-5-yl | 464.18 | 1.47 |
| 87 | — | 3-methoxy-2-methyl-phenyl | 466.19 | 1.65 |
| 88 | — | 2-hydroxy-5-methyl-phenyl | 452.18 | 1.52 |
| 89 | — | 1H-indol-6-yl | 461.18 | 1.52 |
| 90 | — | 1H-indol-5-yl | 461.18 | 1.52 |
| 91 | — | 2-methyl-5-propyl-2H-pyrazol-3-yl | 468.22 | 1.5 |
| 92 | cyclopropyl | phenyl | 462.20 | 1.68 |

### EXAMPLE 93

### [4-(8-Chloro-5-methyl-5,6-dihydro-4H-2,3,5,10b-tetraaza-benzo[e]azulen-1-yl)-piperidin-1-yl]-(3-methoxy-phenyl)-methanone

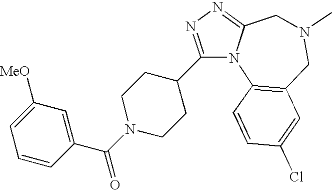

To a solution of 3-methoxybenzoic acid (72 mg, 0.47 mmol) in dichloromethane (10 ml) was added HBTU (O-benzotriazol-1-yl-N,N,N′,N′-tetramethyluronium hexafluorophosphate, 180 mg, 0.47 mmol), followed 30 minutes later by the amine of preparation 10 (VII″) (100 mg, 0.315 mmol). The reaction mixture was stirred at room temperature overnight. Dimethylformamide (2 ml) was added to help solubilisation and further 3-methoxybenzoic acid (48 mg, 0.315 mmol) and HBTU (119 mg, 0.315 mmol) were added. The reaction mixture was stirred at room temperature overnight. It was then partitioned between dichloromethane and an aqueous solution of saturated sodium carbonate. The organic layer was collected, evaporated under reduced pressure and purified by column chromatography on silica gel eluting with dichloromethane:methanol:ammonia (95:5:0.5 to 90:10:1v:v:v) to provide the title compound (55 mg, 39%).

¹H NMR (400 MHz, CDCl₃): δ 1.94-2.05 (brd, 4H), 2.48 (s, 3H), 3.05 (brs, 2H,), 3.18 (m, 1H), 3.32 (brs, 2H), 3.67 (brs, 2H), 3.82 (s, 3H), 3.93 (brs, 1H), 4.64 (brs, 1H), 6.94 (m, 3H), 7.29 (m, 2H), 7.51 (m, 2H); LRMS: m/z APCI⁺, 474 [MNa]⁺

### EXAMPLE 94

### [4-(8-Chloro-5-methyl-5,6-dihydro-4H-2,3,5,10b-tetraaza-benzo[e]azulen-1-yl)-piperidin-1-yl]-(3-fluoro-phenyl)-methanone

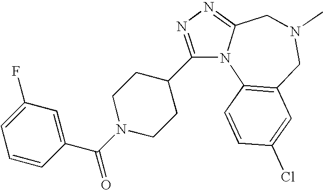

To a solution of the amine from preparation 10 (VII″) (110 mg, 0.35 mmol), in dichloromethane (10 ml), was added triethylamine (73 μl, 0.52 mmol), followed by 3-fluorobenzoyl chloride (73 μl, 0.49 mmol). The reaction mixture was stirred at room temperature overnight. It was then washed with an aqueous solution of saturated sodium carbonate. The organic layer was collected, evaporated under reduced pressure and purified by column chromatography on silica gel eluting with dichloromethane:methanol:ammonia (90:10:1v:v:v) to provide the title compound (52 mg, 34%).

¹H NMR (400 MHz, CDCl₃): δ 1.99 (brs, 4H), 2.48 (s, 3H), 3.07 (brs, 2H,), 3.18 (m, 1H), 3.32 (brs, 2H), 3.66-3.88 (brs, 3H), 4.59 (brs, 1H), 7.11 (m, 2H), 7.18 (m, 1H), 7.28 (m, 1H), 7.36 (m, 1H), 7.51 (m, 2H); LRMS: m/z APCI⁺, 440 [MH]⁺

### EXAMPLE 95

### [4-(8-Chloro-5-methyl-5,6-dihydro-4H-2,3,5,10b-tetraaza-benzo[e]azulen-1-yl)-piperidin-1-yl]-(4-fluoro-phenyl)-methanone

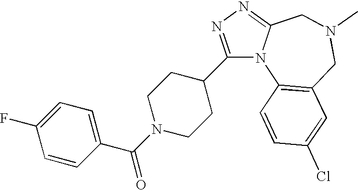

The title compound was prepared by a method similar to that described for example 94 using the amine of preparation 10 (VII″) and 4-fluorobenzoyl chloride.

¹H NMR (400 MHz, CDCl₃): δ 1.98 (brs, 4H), 2.48 (s, 3H), 3.06 (brs, 2H,), 3.18 (m, 1H), 3.32 (brs, 2H), 3.67 (brs, 2H), 4.07 (brs, 1H), 4.51 (brs, 1H), 7.09 (t, 2H), 7.29 (m, 1H), 7.41 (m, 2H), 7.52 (m, 2H); LRMS: m/z APCI⁺, 440 [MH]⁺

### EXAMPLE 96

### 1-[4-(8-Chloro-5-methyl-5,6-dihydro-4H-2,3,5,10b-tetraaza-benzo[e]azulen-1-yl)-piperidin-1-yl]-butan-1-one

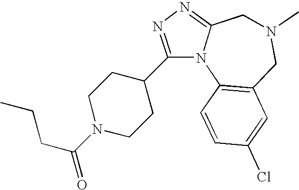

The title compound was prepared by a method similar to that described for example 94 using the amine of preparation (VII″) and butyryl chloride.

¹H NMR (400 MHz, CDCl₃): δ 0.96 (t, 3H), 1.65 (sex, 2H), 1.82-2.02 (brm, 4H), 2.30 (m, 2H), 2.49 (s, 3H), 2.75 (brs, 1H,), 3.12 (m, 2H), 3.34 (brs, 2H), 3.68 (brs, 2H), 3.98 (brd, 1H), 4.52 (brs, 1H), 7.26 (m, 1H), 7.52 (m, 2H); LRMS: m/z APCI⁺, 388 [MH]⁺

### EXAMPLE 97

### [4-(8-Chloro-5-methyl-5,6-dihydro-4H-2,3,5,10b-tetraaza-benzo[e]azulen-1-yl)-piperidin-1-yl]-cyclopropyl-methanone

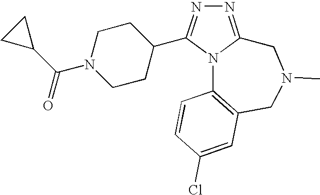

The title compound was prepared by a method similar to that described for example 94 using the amine of preparation (VII″) and cyclopropane carbonyl chloride.

¹H NMR (400 MHz, CDCl₃): δ 0.75 (m, 2H), 0.97 (m, 2H), 1.74 (m, 1H), 1.86-2.03 (brm, 4H), 2.48 (s, 3H), 2.80 (brs, 1H,), 3.13 (m, 2H), 3.33 (brm, 2H), 3.66 (brs, 2H), 4.33 (brd, 1H), 4.48 (brs, 1H), 7.29 (d, 1H), 7.51 (m, 2H); LRMS: m/z APCI⁺, 386 [MH]⁺

All of the compounds exemplified above showed a Ki value of less than 500 nM when tested in screen 1.0 (V_{1A} filter binding assay) as described above. Examples of specific compounds are illustrated in Table 2 below.

TABLE 2

| Example No. | Ki (nM) |
|---|---|
| 1 | 73.76 |
| 2 | 35.00 |
| 9 | 59.92 |
| 18 | 62.14 |
| 64 | 34.68 |
| 67 | 15.89 |

1. A compound of formula (I), 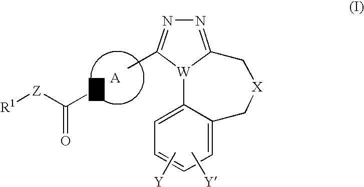
or a pharmaceutically acceptable derivative thereof, wherein:
X represents NR or O;
R represents hydrogen, C₁₋₈ alkyl or SO₂[C₁₋₈ alkyl];
W represents N or CH;
Y and Y′ independently represent hydrogen, halogen, OH, CF₃, OCF₃, CN, NH₂, C₁₋₈ alkyl, C₁₋₈ alkyloxy or C₃₋₈ cycloalkyl;
Ring A represents a heterocyclic ring containing at least one nitrogen atom;
Z represents a direct link, C₁₋₈ alkyl or C₃₋₈ cycloalkyl;
R¹ represents R², OR², OR³—R⁴, N(R²)[C₁₋₈ alkylene]ₐR⁴; NCOR², or SR⁴;
R² and R⁴ independently represent hydrogen, C₃₋₈ cycloalkyl, CF₃, Ar or Het;
R³ represents a direct link or C₁₋₈ alkyl;
a is 0 or 1;
Ar represents an aromatic ring, optionally fused to a heterocyclic ring, and/or optionally substituted with one or more groups as described below;
Het represents a heterocyclic ring optionally substituted with one or more groups as described below, and/or optionally fused to an aromatic ring which is optionally substituted with one or more groups as described below;
at each occurrence C₁₋₈alkyl, C₁₋₈alkylene and C₃₋₈cycloalkyl may be independently optionally substituted with one or more groups as described below;
substituent groups for Ar, Het, C₁₋₈alkyl, C₁₋₈alkylene and C₃₋₈cycloalkyl referred to above are independently selected from hydrogen, halogen, C₁₋₈alkyl, C₁₋₈alkyloxy, S[C₁₋₈alkyl], CN, CF₃, NH₂ and OH.

2. A compound according to claim 1, wherein X represents NR and R represents Me.

3. A compound according to claim 1 or claim 2, wherein W represents N.

4. A compound according to any of claims 1 to 3, wherein Ring A represents piperidinyl.

5. A compound according to any of claims 1 to 4, wherein Z is a direct link.

6. A compound according to claim 1, selected from
[4-(8-Chloro-5-methyl-5,6-dihydro-4H-2,3,5,10b-tetraaza-benzo[e]azulen-1-yl)-piperidin-1-yl]-(1H-indol-3-yl)-methanone;
1-[4-(8-Chloro-5-methyl-5,6-dihydro-4H-2,3,5,10b-tetraaza-benzo[e]azulen-1-yl)-piperidin-1-yl]-2-o-tolyl-ethanone;
[4-(8-Chloro-5-methyl-5,6-dihydro-4H-2,3,5,10b-tetraaza-benzo[e]azulen-1-yl)-piperidin-1-yl]-(1-methyl-cyclohexyl)-methanone;
1-[4-(8-Chloro-5-methyl-5,6-dihydro-4H-2,3,5,10b-tetraaza-benzo[e]azulen-1-yl)-piperidin-1-yl]-2-cyclopropyl-ethanone;
[4-(8-Chloro-5-methyl-5,6-dihydro-4H-2,3,5,10b-tetraaza-benzo[e]azulen-1-yl)-piperidin-1-yl]-(1H-indol-2-yl)-methanone;
[4-(8-Chloro-5-methyl-5,6-dihydro-4H-2,3,5,10b-tetraaza-benzo[e]azulen-1-yl)-piperidin-1-yl]-(2-hydroxy-5-methyl-phenyl)-methanone;
[4-(8-Chloro-5-methyl-5,6-dihydro-4H-2,3,5,10b-tetraaza-benzo[e]azulen-1-yl)-piperidin-1-yl]-(1H-indol-6-yl)-methanone;
[4-(8-Chloro-5-methyl-5,6-dihydro-4H-2,3,5,10b-tetraaza-benzo[e]azulen-1-yl)-piperidin-1-yl]-(3-methoxy-phenyl)-methanone;
[4-(8-Chloro-5-methyl-5,6-dihydro-4H-2,3,5,10b-tetraaza-benzo[e]azulen-1-yl)-piperidin-1-yl]-(3-fluoro-phenyl)-methanone;
[4-(8-Chloro-5-methyl-5,6-dihydro-4H-2,3,5,10b-tetraaza-benzo[e]azulen-1-yl)-piperidin-1-yl]-(4-fluoro-phenyl)-methanone;
1-[4-(8-Chloro-5-methyl-5,6-dihydro-4H-2,3,5,10b-tetraaza-benzo[e]azulen-1-yl)-piperidin-1-yl]-butan-1-one;
[4-(8-Chloro-5-methyl-5,6-dihydro-4H-2,3,5,10b-tetraaza-benzo[e]azulen-1-yl)-piperidin-1-yl]-cyclopropyl-methanone; and
pharmaceutically acceptable derivatives thereof.

7. The use of a compound according to any of claims 1 to 6 as a medicament.

8. A method of treatment of anxiety, cardiovascular disease (including angina, atherosclerosis, hypertension, heart failure, edema, hypernatremia), dysmenorrhoea (primary and secondary), endometriosis, emesis (including motion sickness), intrauterine growth retardation, inflammation (including rheumatoid arthritis), mittelschmerz, preclampsia, premature ejaculation, premature (preterm) labor or Raynaud's disease, comprising administering a therapeutically effective amount of a compound according to any of claims 1 to 6 to a patient suffering from such a disorder.

9. A method according to claim 7 wherein the disorder is dysmenorrhoea (primary or secondary).

10. A method according to claim 9 wherein the disorder is primary dysmenorrhoea.

11. The use of a compound according to any of claims 1 to 6 in the manufacture of a medicament for the treatment of anxiety, cardiovascular disease (including angina, atherosclerosis, hypertension, heart failure, edema, hypernatremia), dysmenorrhoea (primary and secondary), endometriosis, emesis (including motion sickness), intrauterine growth retardation, inflammation (including rheumatoid arthritis), mittelschmerz, preclampsia, premature ejaculation, premature (preterm) labor or Raynaud's disease.

12. Use according to claim 11 wherein the disorder is dysmenorrhoea (primary or secondary).

13. Use according to claim 12 wherein the disorder is primary dysmenorrhoea.

14. A pharmaceutical formulation including a compound according to any of claims 1 to 6 or a pharmaceutically acceptable derivative thereof, together with a pharmaceutically acceptable excipients, diluent or carrier;

15. A pharmaceutical product containing a V1a antagonist according to any of claims 1 to 6 in combination with a compound selected from (a) an oral contraceptive, (b) a PDE5 inhibitor, (c) an NO donor, (d) L-arginine, or (e) a COX inhibitor, as a combined preparation for simultaneous, separate or sequential use in the treatment of dysmenorrhoea.
